(19) United States
(12) Patent Application Publication
(10) Pub. No.: US 2019/0178957 A1
(43) Pub. Date: Jun. 13, 2019

(54) METHOD OF TESTING PRODUCTS

(71) Applicants: JERRY PACIFIC, Hermosa Beach, CA (US); STEVEN JARSKY, Santa Monica, CA (US)

(72) Inventors: JERRY PACIFIC, Hermosa Beach, CA (US); STEVEN JARSKY, Santa Monica, CA (US)

(21) Appl. No.: 16/323,964

(22) PCT Filed: May 3, 2018
(86) PCT No.: PCT/US18/30883

§ 371 Date: Feb. 7, 2019

Related U.S. Application Data
(60) Provisional application No. 62/500,622, filed on May 3, 2017.

(51) Int. Cl. G01R 33/12; A61N 2/00

(52) U.S. Cl. CPC G01R 33/12; A61N 2/00

(57) ABSTRACT

Testing products to ascertain a type of energy represented by the product, labeling the product, and publishing the results of the test to assist a consumer in determining whether or not to obtain the product. The testing is done by placing a magnet on the product and using the bi-digital O-ring testing process.

### RELATED APPLICATIONS

This is the nonprovisional application based upon Provisional Application Ser. No. 62/500,622, filed May 3, 2017, and Applicant claims the benefits of the first filed filing date thereof.

### FIELD OF THE INVENTION

The present invention relates generally to the testing of products to determine the type of energy represented by the product and more specifically is directed to determining whether or not the product predominantly contains a Yin energy or a Yang energy according to traditional Chinese medicine and labelling the product accordingly to assist users or consumers of the product in determining whether or not to obtain the product.

### BACKGROUND OF THE INVENTION

All products contain energy. According to traditional Chinese medicine, energy is divided into two types, Yin energy and Yang energy. Traditional Chinese medicine views the body as an energetic system in dynamic balance. Qi (pronounced Chee) can be translated as energy or life force and flows in a regular pattern to all parts of the body. When the flow of Qi is unimpeded, there is harmony, balance and good health. When there are Qi blockages, too much or insufficiency of Qi, there is an imbalance which leads to disharmony.

The primary goal of traditional Chinese medicine is to create wholeness and harmony within a person allowing the mind/body/spirit to heal itself.

Traditional Chinese medicine theory postulates that it is the internal ability of the body to remain strong. That is the key to health. In this theory, people are born with a certain amount of original Qi which is easily depleted as energy is used by the body and not replaced. Original Qi cannot be increased but only maintained through staying as balanced as possible.

A balanced lifestyle system in accordance with traditional Chinese medicine is composed of everything that creates and sustains it. Everything is interconnected and interdependent if all of the parts of the system are in harmony with one another, then the whole system is in balance. Qi is energy in the very broadest sense possible and is universal. Qi embraces all forms of energy from the most material aspects of energy including all products which are by definition constructed of matter which takes various forms.

Because the dynamics of Yin and Yang are always shifting and changing in the world around us, we must continuously respond to these changes in order to maintain the balance of Yin and Yang in our lives and so maintain our health and wellbeing. If we do not make appropriate responses to change, then the harmony of Yin and Yang in our life may tilt to a pattern of disharmony. The potential causes of disharmony fall into two general categories—deficiency and excess.

Deficiency usually refers specifically to a variety of one or more of the manifestations of Qi within the body, but it can also refer more generally to an insufficiency of any of the things that would normally sustain and nourish the body. To make an appropriate response to the ever-shifting dynamics of Yin and Yang we must have a sufficient amount of Qi. Since most of the Qi we use in life is extracted from what we eat and the air that we breath, traditional Chinese medicine places considerable importance upon adequate and appropriate diet.

Excess usually refers to the presence of something that is in some way replete to our nature but can also refer to an overabundance of something relative to our need for it.

To avoid these imbalances caused by deficiency or excess, we must create a lifestyle incorporating Yin and Yang and we must have sufficient mindfulness and mental clarity to perceive the world and determine our needs and we must determine how we can meet those needs without creating new problems for ourselves or creating disharmony in the world around us.

Since matter contains energy and all products consist of matter in one form or another, it is very important that the products that are used or consumed by human beings or animals are categorized into the appropriate energy in such a manner that the utilization or consumption of these products by human beings or animals which contain Qi, help to support the proper balance of Yin and Yang energy, which maintains harmony. To obtain the proper balance, an object of the present invention is to maximize the amount of energy to the person involved, that is, more Yin for women and more Yang for men. It is, therefore, essential that all of the products which are to be used or consumed by human beings or animals are properly labeled according to the energy which they possess and that such products be labeled accordingly to assist human beings in determining which products to acquire and use or consume in their normal day-to-day lives.

### SUMMARY OF THE INVENTION

The present invention is a method of classifying products for use or consumption which includes testing a specific product to determine whether it provides a stronger or weaker energy effect to users or consumers of that specific product. By placing the product in contact with a human and placing a magnet in contact with the product and applying the bi-digital O-ring test, labeling the specific product according to the results of the test, publishing the test results and labeling the product to assist the consumer in making decisions as to whether or not to obtain the product for use or consumption.

### BRIEF DESCRIPTION OF THE DRAWINGS

FIGS. 1 and 2 illustrate the bi-digital O-ring test;

FIG. 3 is a schematic illustration showing the testing of a product in accordance with the principles of the present invention; and

FIG. 4 illustrates the testing of a product when an individual is too weak to participate.

### DETAILED DESCRIPTION OF THE PREFERRED EMBODIMENT

It has been determined that energy circuits exist in organisms such as the human body and that such energy fields extend to within five centimeters around the body. As above described, all products are made of matter and matter has energy and, as a result, that energy creates an electromagnetic field which exists around the particular product. The present invention takes advantage of the existence of the electromagnetic fields which interact between the products to be tested and the human body. The testing of the products is accomplished in accordance with a preferred embodiment of the present invention though the utilization of the bi-digital O-ring test. The bi-digital O-ring test is disclosed in detail in the Omura U.S. Pat. No. 5,188,107 and by this reference is incorporated herein. As above indicated in classifying a particular product, it is placed in contact with a human being and a magnet is applied to the product and the tester then utilizes the bi-digital O-ring test to determine whether or not the product is Yin energy or Yang energy.

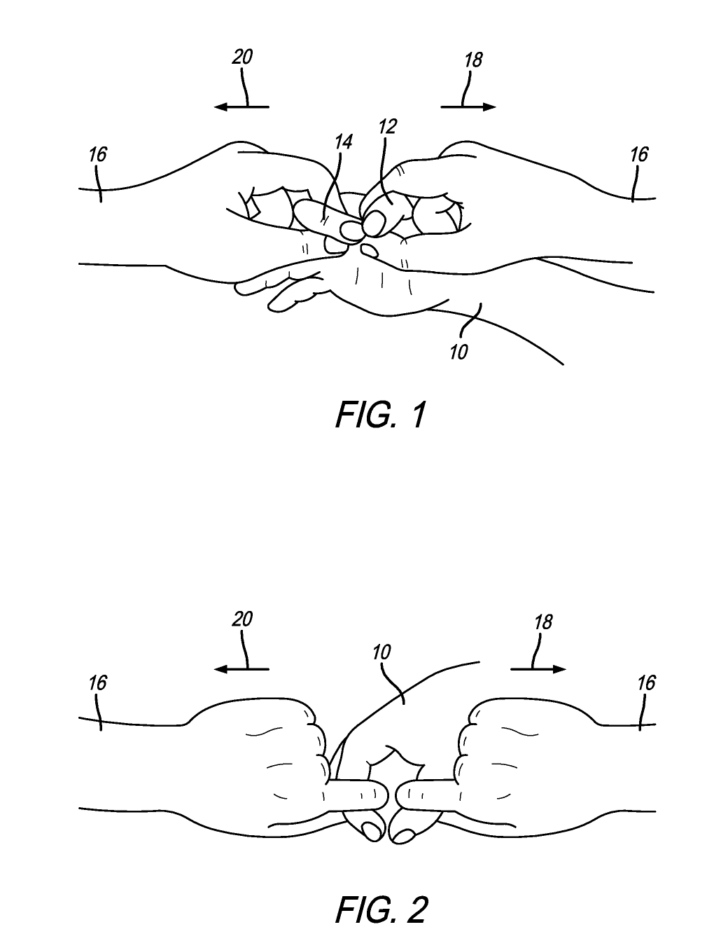

As shown in FIG. 1, the individual who is being utilized in the test is shown at 10. The individual providing the bi-digital O-ring test brings his thumb and a finger on the same hand together as shown at 12 and 14. The individual performing the test who is shown at 16 will then interlock fingers on both of that individual's hands internally of the O-ring formed by the human 10 and as is illustrated in FIG. 1, such is done by bringing together the index finger and the thumb of each hand and then, as shown by the arrows 18 and 20, the tester 16 will exert a force to try to separate the bi-digital O-ring of the human being 10. Although the thumb and index finger of the human being 10 is shown in FIGS. 1 and 2, it should be understood that the thumb may be brought into contact with any of the other fingers which may be desired depending upon the relative strengths of the human who is being utilized in the test and the tester and the predominant Yin or Yang strength level of the product.

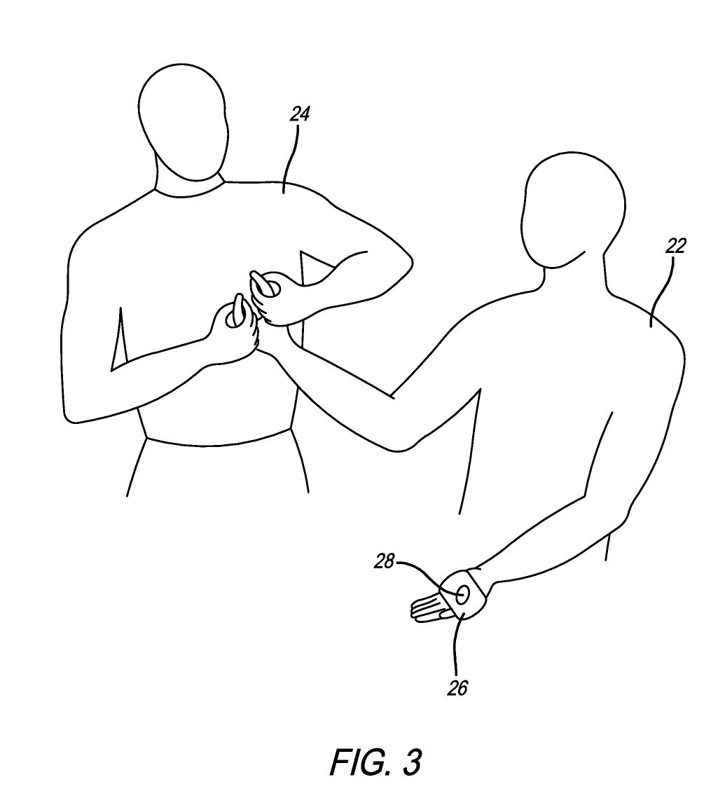

Referring now more specifically to FIG. 3, there is a schematic illustration that illustrates the manner in which a product is tested in accordance with the principles of the present invention. As is shown in FIG. 3, the human being utilized in the test is illustrated at 22 and the tester is illustrated at 24. In accordance with the principles of the present invention, a product being tested such as illustrated at 26 is placed in contact with the human being 22. In this instance, it is illustrated that the product 26 is placed in the palm of the hand of the human being 22. However, it should be understood that the product 26 may be placed in contact with the human being in other positions on any other part of the body of the human being 22. As is described above, the invention is testing any product of any kind which may be used or consumed by a human being or an animal, however, for purposes of discussion and simplicity in describing the present invention, a product 26 will be described as a food product. As above described, the food product does contain energy and that energy in accordance with traditional Chinese medicine is divided into two types, Yin energy and Yang energy. Yang energy is male, warming and moving. Yin energy is female, cooling and nourishing. Magnets also have two types of energies or poles. There is a North pole and a South pole. The North pole of a magnet is Yin and the South pole of the magnet is Yang. As shown in FIG. 3, a magnet 28 is placed on the food product 26. The magnet has a North pole and a South pole. The North pole of the magnet is Yin and the South pole of the magnet is Yang. If the magnet 28 is placed on the food product 26 with the South pole (Yang) facing down onto the food product 26, a weak response will be created if the food product 26 has predominantly Yang energy. Such occurs because the South pole of the magnet is Yang and the food product has Yang energy. This would then be the same as placing two South pole magnets together which will illicit an opposing force. The same is true when testing a product such as the food product. If the magnet 28 is turned over and the food product 26 is a Yang product, then there would be a strong attractive energy experienced.

As is shown in FIG. 3, assuming that the food product has predominantly Yang energy and the South pole of the magnet 28 is placed downwardly on top of the food product 26, then the tester would be able to easily pull apart the bi-digital O-ring as is show in FIG. 3. As above described, then if the magnet is turned over and a strong attractive energy is created, the tester 24 would not be able to separate the bi-digital O-ring of the human being 22 unless excessive force would be applied to the thumb and finger of the human being 22. In this manner, it could easily be determined that the product 26, in this case the food product, has predominantly Yang energy and that food product would be so labeled to provide a customer with that type of information so that the customer could then recognize that if the food product 26 is purchased and consumed it would provide strong male Yang energy to the system of the human being.

Likewise if the food product 26 with the magnet 28 placed so that the South pole is down and the tester 24 has a difficult time separating the bi-digital O-ring of the human being 22, it would then be determined that the product 26 is one having predominantly Yin energy and the product would be so labeled to assist the consumer in purchasing the product.

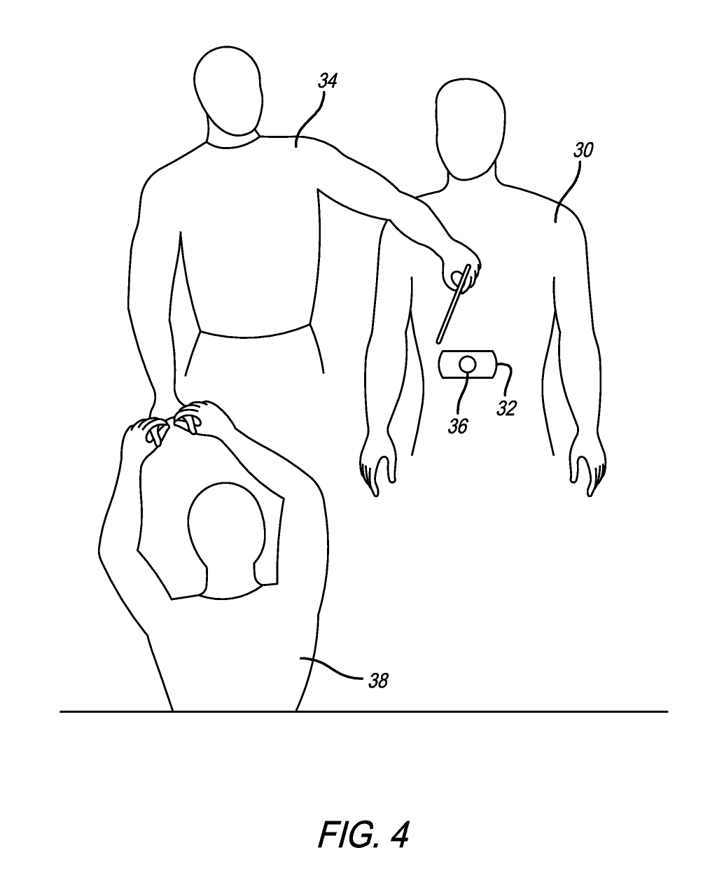

In the event that using the bi-digital O-ring in a manner as above described is not possible, such as with a child or animal, or a weaker human being, then the method could be also utilized by including a non-invasive external probe utilizing a non-conductive probing rod-type device such as that made of glass which is placed on the external area of the human being as illustrated in FIG. 4.

As shown in FIG. 4, the individual 30 is too weak or is a child and is unable to provide the required digital O-ring. In such a case, the item 32 to be tested is placed on the individual 30 and a third person 34 places the noninvasive, non-conductive probe on the external area of the human being with the magnet 36 in contact with the product 32 thereon. The third party 34 forms the bi-digital O-ring as above described and the tester 38 applies the force to part the bi-digital O-ring and determine whether the item 32 is predominantly Yin or Yang energy.

There has thus been disclosed a method for testing a product to determine whether or not that product has predominantly Yin or Yang energy in accordance with traditional Chinese medicine and then to be able to label the product accordingly to assist the consumer in making the decision to purchase the product.

1. A method of classifying products for use or consumption comprising:
testing the specific product to determine whether it provides a positive or negative effect to users or consumers of that specific product;
labeling the specific product according to the results of the test;
publishing the test results and the labeling for use by consumers to assist the consumer in making decisions to purchase products; and
providing such labeled products for purchase by consumers.

2. The method as defined in claim 1 wherein the testing step comprises placing the specific product in contact with a human being, placing a magnet in contact with the product with one of the North and South poles of the magnet facing the product, and applying the bi-digital O-ring test.

3. The method as defined in claim 2 which comprises the further step of reversing the magnet to have the other of the North and South poles of the magnet facing the product.

4. The method as defined in claim 2 which comprises the further step of a third person placing a non-invasive probe on the external area of the human being and forms the bi-digital O-ring.

5. The method as defined in claim 2 wherein the product is a food product.
